(19) United States
(12) Patent Application Publication
(10) Pub. No.: US 2006/0099694 A1
(43) Pub. Date: May 11, 2006

(54) Process for increasing the yield of lipid and omega-3 fatty acid in seaweed culture

(75) Inventors: Rejean Tremblay, Grande-Riviere (CA); Fabrice Pernet, Carleton (CA); Edwin Bourget, Ascot (CA)

Correspondence Address: David S. Resnick;Nixon Peabody, 100 Summer Street, Boston, MA, 02110-2131, US

(73) Assignees: Universite de Sherbrooke, Sherbrooke, QC (CA); Universite Laval, Quebec, QC (CA); Rival S.E.C., Montreal, QC (CA)

(21) Appl. No.: 10/521,868

(22) PCT Filed: Jul. 22, 2003
(86) PCT No.: PCT/CA03/01100

§ 371 Date: Aug. 30, 2005

(30) Foreign Application Priority Data
Jul. 22, 2002 (CA) 2,395,622

(51) Int. Cl. C12P 7/64; C12N 1/12

(52) U.S. Cl. 435/134; 435/257.1

(57) ABSTRACT

The present invention relates to a new method for producing polyunsaturated fatty acids from algae. The method comprises the step of applying at least growth-limiting factor to an algae culture, causing growth arrest of said algae culture and production and stocking by algae in culture of polyunsaturated fatty acids.

### BACKGROUND OF THE INVENTION

(a) Field of the Invention

The present invention relates to a new process for producing polyunsaturated fatty acid (PUFAs) and more particularly for producing omega-3.

(b) Background of the Invention

Microalgae, and more particularly those cultured in a mariculture, are often rich in PUFAs, among which the two most important species are eicosapentanoic acid (EPA) and docosahexanoic acid (DHA). Table 1 below shows concentrations of EPA and DHA of various species of microalgae maintained in standard culture.

TABLE 1
FATTY ACID OF VARIOUS SPECIES OF MICROALGAE

% Fatty acid

| | EPA | DHA |
|---|---|---|
| Chrysophyceae | | |
| Pseudopedinella | 27 | 1 |
| Circosphaera | 28 | — |
| Isochrysis | — | 15 |
| Xanthophyceae | | |
| Nannochloris | 27 | — |
| Bacillariophyceae | | |
| Nitzchia | 17 | — |
| Phaedactylum tricornatum | 28 | — |
| Rhodophyceae | | |
| Porphyridium cruentum | 17 | — |
| Dinophyceae | | |
| Amphidinium carteraem | 20 | 24 |
| Ceratium furca | 7 | 21 |
| Cochlodinium spp. | 11 | 28 |
| Crypthecodinium cohnii | — | 30 |
| Gonyaulax spp. | 12-34 | 1-16 |
| Peridinium triquetum | 19 | 2 |
| Procentrum spp. | 15-32 | 3-5 |

References: W. Yongmanitchai and O. P. War (1989; Omega-3 fatty acids: alternative sources of production; Proc. Biochem 24: 117-125) and J. K. Volman et al. (1989; Fatty acid and lipid composition of 10 species of microalgae used in mariculture; J. Exp. Mar. Biol. Ecol. 128: 219-240

Mariculture of microalgae for producing PUFAs has been set up originally with only those species that are known to be rich in fatty acid, such as Crypthecodinium cohnii.

Lipid content such as PUFAs of microalgae will vary depending on their culture conditions. However, the conditions that would be optimal for obtaining this concentration of fatty acid in algae are incomparable with those necessary for the growth of the algae in a culture. Accordingly, a culture of algae rich in a lipid such as a fatty acid can only be carried out at a low concentration.

Accordingly, it would be advantageous to be provided with a process for producing PUFAs at a high concentration, allowing for reduction of the culture volume for obtaining the same yield of PUFAs.

### SUMMARY OF THE INVENTION

One aim of the present invention is to provide a new process for producing PUFAs, and to obtain a high concentration of a lipid.

In accordance with the present invention, there is provided a process for producing PUFAs by blocking cell division, and thus culture growth, allowing to obtain a lipid-rich culture.

Still in accordance with the present invention, there is provided a method for producing polyunsaturated fatty acids from algae, comprising the step of applying at least growth-limiting factor to an algae culture, causing division arrest of said algae culture and production and stocking by algae in culture of polyunsaturated fatty acids.

The growth-limiting factor may be for example silicate deprivation other nutrient deprivation or physical factors such as light intensity for example. In one embodiment of the invention, more than one growth-limiting factor can be applied either simultaneously or concurrently. Preferred algae for carrying out the method of the present invention are diatomaceous Chaetoceros gracilis or diatomaceous Skeleonema costatum.

In one embodiment of the invention, the growth-limiting factor is applied at the end of the exponential growth phase, and preferably when the algae culture has reached a concentration of at least 10⁷ cells/mL. Blocking cell division of the algae in culture (and thus growth of the culture) at that specific point in time allows obtaining algae that are rich in PUFAs, and more particularly in omega-3 fatty acid.

### DETAILED DESCRIPTION OF THE INVENTION

Algae are cultured in a semi-continuous process at a temperature, a pH and illumination conditions adapted for their growth. More particularly, the algae are preferably cultured at a temperature of 18 to 20° C., a pH of 7.5 to 8.0 and lighting condition from only one side of the culture flask. The light was provided by Cool-white™ and Growlite™ fluorescent lights at an intensity varying from 60 to 250 μE s⁻¹ m⁻². The photoperiod has a 16-hour lighting cycle followed by 8 hours of darkness. Water used for the cultures was filtered at 1 μm and pasteurized at 80° C.

In preliminary testing, 2-3 ml of original algae inoculums were added to 125 ml erlenmeyers containing 75 ml of f/2 culture medium (Guillard, R., 1975; Culture of phytoplankton for feeding marine invertebrates. In: Smith, W. L., Chanley, M. H. (Eds.), Culture of marine invertebrates animals. Plenum Press, New York, pp. 29-60). Seven days after inoculation, the content of the erlenmeyers was transferred in a 500-ml erlenmeyers container containing 300 ml of f/2 culture media. Five days later, the content of the 500-ml erlenmeyers was transferred into a 20-litre culture bottle. During the cultures in 125 and 500 ml erlenmeyers, no specific additional element or nutrient has been added to the cultures.

In the 20-litre culture bottle, 8 ml of f/2 culture media was added with 18 litres of water. After two days, 4 ml of silicate was added and, after three additional days, the content of the 20-litre culture bottle was transferred to a 7-feet high, 170-litre culture tube. 62 ml of f/2 culture media and 31 ml of silicate were then added to the tubes which were then filled up with water. Nutrients, with or without silicate, according to the species grown, were added every other day. In the 20-litre and 170-litre culture bottle or tubes, filtered air and CO₂ is added at a rate of 0.2 to 0.3 L/min.

After 6-7 days of incubation in the 170-litre tube, the algae cultures were at the end of their exponential growth phase, and have thus attained a maximum concentration. Only by the end of the exponential growth phase were the algae stressed by depriving them of nutrients in order to modify/alter their metabolism. The algae, in reaction to the stress, stop dividing and start stocking up lipids, mostly PUFAs. The exact nature of the nutritional or environmental stress imposed on the algae will depend on the species being cultured. For certain species, concentrations of PUFAs were almost doubled when compared to identical algae cultures that were not nutrient-deprived.

In accordance with the present invention, it was found that imposing stress on the algae culture would cause the algae to stop growing and to start stocking up lipids, mostly PUFAs. Various types of stress could be imposed on the algae culture, such as nutritional stress during which the cell culture is deprived of nutrients, or environmental stress during which the pH and/or lighting conditions are modified so as to cause the algae to stop growing/dividing. Preferably, stress is imposed on the algae once these have completed their exponential growth phase, at which time the concentration of algae in the culture is optimal. One skilled in the art will have no difficulty understanding that in order to obtain as much lipid as possible, it is thus desirable to have a maximum concentration of algae that would, in turn, produce a maximum concentration of lipid. However, in the present invention, it is demonstrated that nutrient depriving or otherwise stressing an algae culture will cause the algae to stop growing/dividing and start stocking up lipids.

In accordance with the present invention, various species of algae have been tested and it has been shown that the method of the present invention does indeed apply and that lipid rich algae cultures are obtainable. However, one skilled in the art will well appreciate that various algae species can have the same modification in the metabolic process, i.e. cell division arrest, that would provide the same significant increase in PUFAs.

The present invention will be more readily understood by referring to the following examples which are given to illustrate the invention rather than to limit its scope.

### EXAMPLE 1

Diatomaceous Chaetoceros gracilis was cultured in a semi-continuous system of 170 litres, at concentrations of more than 10⁷ cells/ml. Some of the tubes were supplemented with complete nutrients whereas other tubes were silicate deprived. The results as reported in Table 2 hereinbelow show the distribution of fatty acids according to the treatment.

TABLE 2
CONCENTRATION OF VARIOUS FATTY ACIDS OBTAINED IN
DIFFERENT CULTURE CONDITIONS

| | With silicate % | Without silicate % |
|---|---|---|
| 20:5n3 | 8.9 | 30.2 |
| 22:6n3 | 3.9 | 8.5 |
| Total PUFA | 33.1 | 50.0 |
| Total n3 | 21.1 | 34.9 |

The analysis of the culture condition was carried out 7 days after the stress (silicate deprivation) was initiated.

### EXAMPLE 2

Diatomaceous Skeletonema costatum was cultured in a semi-continuous system of 170 litres. Some of the tubes were deprived in silicate whereas other tubes were maintained with the complete nutrients. The results represented in Table 3 hereinbelow show the distribution of various fatty acids according to the stress imposed.

TABLE 3
DISTRIBUTION OF VARIOUS FATTY ACIDS IN RESPONSE
TO SILICA DEPRIVATION

| | With silicate % | Without silicate % |
|---|---|---|
| 20:5n3 | 16. | 37.6 |
| 22:6n3 | 5.5 | 7.54 |
| Total PUFA | 41.0 | 59.9 |
| Total n3 | 24.6 | 42.0 |

Here again, the analysis of the culture condition was carried out 7 days after the silicate deprivation was initiated.

The above examples were given here to demonstrate and not to limit the present invention. It is being demonstrated herein that, in accordance with the present invention, it was possible to increase the yield in lipids and more particularly in PUFAs and omega-3, upon stressing an algae culture causing its division arrest, and thus its growth decrease. With the stress, unicellular cells of microalgae decrease the division and increase the lipid yield.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for producing polyunsaturated fatty acids from diatomaceous Chaetoceros gracilis, comprising the step of applying at least one growth-limiting factor to a diatomaceous Chaetoceros gracilis culture at the end of the exponential growth phase, causing growth arrest of said culture and production and stocking by algae in culture of polyunsaturated fatty acids.

2. A method for producing polyunsaturated fatty acids from diatomaceous Skeleonema costatum, comprising the step of applying at least one growth-limiting factor to a diatomaceous Skeleonema costatum culture at the end of the exponential growth phase, causing growth arrest of said culture and production and stocking by algae in culture of polyunsaturated fatty acids.

3. The process of claim 1, wherein the growth-limiting factor is silicate deprivation.

4. The process of claim 1, wherein the growth-limiting factor is a nutrient deprivation.

5. The process of claim 1, wherein more than one growth-limiting factor is applied.

6. The process of claim 1, wherein the growth limiting factor is applied once the culture has reached a concentration of at least 10⁷ cells/mL.

7. The process of claim 2, wherein the growth-limiting factor is silicate deprivation.

8. The process of claim 2, wherein the growth-limiting factor is a nutrient deprivation.

9. The process of claim 2, wherein more than one growth-limiting factor is applied.

10. The process of claim 2, wherein the growth limiting factor is applied once the culture has reached a concentration of at least 10⁷ cells/mL.
